(19) United States
(12) Patent Application Publication
(10) Pub. No.: US 2013/0261693 A1
(43) Pub. Date: Oct. 3, 2013

(54) SLEEP APNEA TREATMENT SYSTEM

(71) Applicant: Yossi Gross, Moshav Mazor (IL)

(72) Inventor: Yossi Gross, Moshav Mazor (IL)

(73) Assignee: RAINBOW MEDICAL LTD., Herzliya (IL)

(21) Appl. No.: 13/885,360

(22) PCT Filed: Nov. 10, 2011
(86) PCT No.: PCT/IL11/00870

§ 371 Date: Jun. 10, 2013

Related U.S. Application Data
(63) Continuation of application No. 12/946,246, filed on Nov. 15, 2010, now abandoned.

(51) Int. Cl. A61N 1/36

(52) U.S. Cl. CPC A61N 1/36078
USPC 607/42

(57) ABSTRACT

Apparatus is provided for treating obstructive sleep apnea, including an external device (24) configured to be incorporated into a household item which further includes: a breathing sensor (28) configured for placement outside of a body of a subject and to sense activity associated with breathing of the subject and a power supply unit (27) configured to power the external device. A control unit (26) coupled to the breathing sensor is configured to generate a signal (32) in response to the sensed activity associated with breathing. A sublingual implant (34) includes a wireless receiver (40) for receiving the signal, and two or more electrodes (42,44) and circuitry (46) coupled to the electrodes. The circuitry is configured to drive the electrodes to apply current to a tissue of the subject in response to the signal, in a plurality of successive periods of inspiration of the subject, even in the absence of a detection of apnea by the control unit.

### CROSS-REFERENCES TO RELATED APPLICATIONS

The present patent application claims the priority of, and is a continuation-in-part of, U.S. patent application Ser. No. 12/946,246 to Gross, filed Nov. 15, 2010, which is incorporated herein by reference.

### FIELD OF EMBODIMENTS OF THE INVENTION

Some applications of the present invention generally relate to implanted medical apparatus with an external power and control source. Specifically, some applications of the present invention relate to apparatus and methods for treating sleep apnea, particularly, obstructive sleep apnea (OSA).

### BACKGROUND

Sleep apnea is a chronic sleep breathing disorder typically characterized by abnormal pauses (apneas) in an individual's breathing, or alternatively, by instances of abnormally low breathing. Each apnea event can range in duration from seconds to minutes. Sleep apnea, although often unrecognized for years by the subject herself, can have long term health effects: Sleep apnea and other sleep breathing disorders are associated with cardiovascular disease, myocardial infarction, high blood pressure, stroke, arrhythmias, diabetes, sleep-deprived driving accidents, and cerebrovascular disease.

In general, there are three forms of sleep apnea: central sleep apnea (CSA), obstructive sleep apnea (OSA), and complex sleep apnea—a heterogeneous group of sleep-related breathing disturbances, with characteristics partially related to both CSA and OSA.

With CSA, cessations in air flow occur without respiratory effort. With OSA (alternatively, obstructive sleep apnea/hypopnea syndrome (OSAHS)), which has overlapping pathogenesis and clinical presentation with CSA, a subject's breathing is interrupted by a physical blockage in the airflow, often characterized, but not limited to snoring and restless sleep. Cardiac output also tends to decrease during apnea events. Snoring, along with other risk factors such as obesity, diabetes or smoking, is often indicative of OSA, particularly when snoring is punctuated by deep gasps.

OSA, the most common form of sleep apnea, is characterized by repetitive collapse and reopening of the upper airway during sleep, resulting in complete or partial blockage of the upper airway during sleep and leading to hypoxemia and hypercapnia. Typically, dilator muscles in the subject's airway stiffen and dilate various regions of the upper airway while the subject is awake. This activity, however, is reduced during sleep, effectively narrowing the upper airway. Of the dilator muscles, the largest is the genioglossus, (the muscle that forms the majority of the tongue).

Current treatments includes positive airway pressure (PAP) therapy, particularly, continuous positive expiratory pressure (CPAP), oral appliances, surgery, including genioglossus advancement, tongue radiofrequency treatment, midline glossectomy, hyoid suspension, maxillomandibular advancement, and lifestyle changes such as positional therapy and weight loss.

### SUMMARY OF EMBODIMENTS

For some applications of the invention, apparatus for treating obstructive sleep apnea comprises a breathing sensor for sensing breathing activity, configured for placement outside of a body of a subject, e.g., in a mattress or an article of clothing. Breathing can be sensed by, for example, sensing electrical activity, breathing-related motions, or via video imaging of the subject's respiration. The apparatus typically also has a control unit, coupled to the breathing sensor, configured to generate signals in response to the sensed activity associated with breathing. The apparatus further typically has a power unit that powers the breathing sensor, the control unit and an implant, e.g., a sublingual implant. The implant typically comprises a wireless receiver for receiving signals, two or more electrodes, circuitry coupled to the electrodes, and one or more coils coupled to the circuitry to wirelessly receive power from the external device. Typically, the circuitry drives the electrodes to apply current to a sublingual muscle site, or in some applications, to a sublingual nerve site, or in further applications, at a phrenic nerve site, of the subject in response to the signal. There is therefore provided, in accordance with an application of the present invention, apparatus for treating obstructive sleep apnea, comprising:

an external device, comprising:
a breathing sensor configured for placement outside of a body of a subject, and to sense activity associated with breathing of the subject; and
a control unit, coupled to the breathing sensor, configured to generate a signal in response to the sensed activity; and an implant, comprising:
a wireless receiver for receiving the signal;
two or more electrodes; and
circuitry coupled to the electrodes, the circuitry configured to drive the electrodes to apply current to an anatomical site of the subject in response to the signal, in a plurality of successive periods of inspiration of the subject, even in the absence of a detection of apnea by the control unit.

In an application, the implant is configured to be implanted sublingually.

In an application, the implant is configured to stimulate a phrenic nerve of the subject.

In an application, the implant is configured to stimulate breathing muscles associated with a phrenic nerve of the subject.

In an application, the external device is configured to be coupled to a mattress.

In an application, the external device is configured to be coupled to an article of clothing.

In an application, the two or more electrodes are configured to be coupled to a sublingual muscle of the subject.

In an application, the implant comprises at least two antennas that are not aligned in a same direction, and wherein the implant is configured to receive the signal via the at least two antennas.

In an application, wherein the breathing sensor is configured to sense a parameter selected from the group consisting of: airway pressure, snoring sounds, snoring motions, mechanical motion, and electrical activity associated with respiration.

In an application, the implant is operative to vary at least one parameter of the applied current, the parameter selected from the group consisting of: a frequency of pulses of the current, an amplitude of pulses of the current, and duration of pulses of the current.

In an application, the implant comprises an implant sensor configured to provide feedback to the external device.

In an application, the control unit is configured to generate the signal during respective inspiratory phases of at least 30% of respiratory periods of the subject in a ten-minute period.

In an application, the external device comprises a power supply unit, which is configured to supply power wirelessly to the implant.

In an application, the implant comprises at least two antennas that are not aligned in a same direction, and wherein the implant is configured to receive the power via the at least two antennas.

In an application, wherein the two or more electrodes are configured to be coupled to a sublingual nerve of the subject.

In an application, the implant comprises a nerve cuff to which the electrodes are coupled.

There is further provided, in accordance with an application of the present invention, a method for treating obstructive sleep apnea, comprising:

identifying a subject as suffering from obstructive sleep apnea; and

in response, implanting an implant that is configured to:
stimulate an anatomical site in a plurality of successive periods of inspiration of the subject, even in the absence of a detection of apnea; and
wirelessly receive energy from an external device, to power the implant.

In an application, implanting the implant comprises implanting the implant in a vicinity of a phrenic nerve of the subject.

In an application, implanting the implant comprises implanting the implant, the implant being configured to stimulate the site in response to an identification of an inspiration period by an external device, and to withhold the stimulation of the site during at least a portion of an expiration period identified by the external device, in response to the identification of the expiration period by the external device.

In an application, the method further includes configuring the external device to sense breathing of the subject and to provide a signal to the implant to cause the implant to directly stimulate the muscle in response to the sensing of the breathing by the external device, during respective inspiratory phases of at least 30% of respiratory periods in an hour.

In an application, implanting the implant comprises implanting the implant at a sublingual site of the subject.

In an application, implanting the implant comprises implanting the implant, the implant being configured to stimulate the sublingual site in response to an identification of an inspiration period by an external device, and to withhold the stimulation of the sublingual site during at least a portion of an expiration period identified by the external device, in response to the identification of the expiration period by the external device.

In an application, implanting the implant comprises sublingually injecting the implant.

In an application, implanting the implant comprises placing the implant in a position to stimulate a sublingual muscle of the subject.

In an application, implanting the implant comprises placing the implant in a position to stimulate a sublingual nerve of the subject.

There is further provided, in accordance with an application of the present invention, a method for using an implant to treat obstructive sleep apnea, comprising:

configuring an external device to:
sense breathing of the subject;
provide a signal to the implant to cause the implant to directly stimulate an anatomical site in response to the sensing of the breathing by the external device, even in the absence of a detection of apnea, during respective inspiratory phases of at least 30% of respiratory periods in a ten-minute period; and
wirelessly transmit energy from the external device to the implant to power the implant.

In an application, providing the signal comprises providing the signal, the external device being configured to provide a signal to the implant to cause the implant to directly stimulate the anatomical site in response to the sensing of the breathing by the external device, even in the absence of a detection of apnea, during respective inspiratory phases of at least 50% of respiratory periods in a ten-minute period;

In an application, configuring the device to provide the signal comprises configuring the device to provide the signal to a sublingual implant.

In an application, configuring the device to provide the signal comprises configuring the device to provide the signal to a phrenic nerve implant.

There is further provided, in accordance with an application of the present invention, a method for treating obstructive sleep apnea, comprising:

identifying a subject as suffering from obstructive sleep apnea; and

in response to the identifying, sublingually implanting a sublingual implant in the subject, the implant being configured to provide feedback regarding breathing of the subject to an external device and to receive from the external device a signal that (a) is generated by the external device in response to the feedback provided by the sublingual implant, and (b) causes the implant to stimulate a sublingual site of the subject.

In an application, the method further includes coupling the external device to an article of clothing.

In an application, the method further includes configuring the external device to sense breathing and to provide the signal in response to the feedback provided by the sublingual implant and in response to the breathing sensed by the external device.

In an application, implanting the sublingual implant configured to provide feedback regarding breathing, comprises implanting a sublingual implant configured to provide feedback regarding snoring of the subject.

In an application, the method further includes juxtaposing the external device to a household item.

In an application, juxtaposing the external device to the household item comprises coupling the external device to the household item.

In an application, juxtaposing the external device comprises juxtaposing the external device to a part of a bed.

There is further provided, in accordance with an application of the present invention, a method for treating obstructive sleep apnea, comprising:

identifying a subject as suffering from obstructive sleep apnea; and

in response to the identifying, implanting a phrenic nerve implant in the subject, the implant being configured to provide feedback regarding breathing of the subject to an external device and to receive from the external device a signal that (a) is generated by the external device in response to the feedback provided by the phrenic nerve implant, and (b) causes the implant to stimulate an anatomical site of the subject in the vicinity of the phrenic nerve.

There is further provided, in accordance with an application of the present invention, a method for treating obstructive sleep apnea of a subject, the method comprising:

extracorporeally detecting breathing of the subject;

automatically extracorporeally generating a signal, at least in part responsively to the detected breathing;

intracorporeally detecting the signal; and

automatically stimulating a site of the subject, at least in part responsively to the signal, the site selected from the group consisting of: a sublingual muscle of the subject and a phrenic nerve of the subject.

In an application, the method further includes extracorporeally detecting reclining of the subject, wherein automatically extracorporeally generating the signal comprises automatically extracorporeally generating the signal at least in part responsively to the reclining of the subject.

In an application, the method further includes intracorporeally detecting reclining of the subject, wherein automatically stimulating the sublingual muscle of the subject comprises automatically stimulating the sublingual muscle of the subject at least in part responsively to the reclining of the subject.

In an application, extracorporeally detecting the breathing comprises extracorporeally detecting the breathing while the subject is sleeping.

In an application, intracorporeally detecting the signal comprises wirelessly receiving power via the signal, using an implant, and wherein automatically stimulating the site of the subject comprises powering the implant using the received power.

The present invention will be more fully understood from the following detailed description of embodiments thereof, taken together with the drawings, in which:

### BRIEF DESCRIPTION OF THE DRAWINGS

FIG. 1 is a schematic illustration of a system including an external device and a sublingual implant, in accordance with some applications of the present invention; and

FIG. 2 is a schematic illustration of the sublingual implant of FIG. 1, in accordance with some applications of the present invention.

### DETAILED DESCRIPTION OF EMBODIMENTS

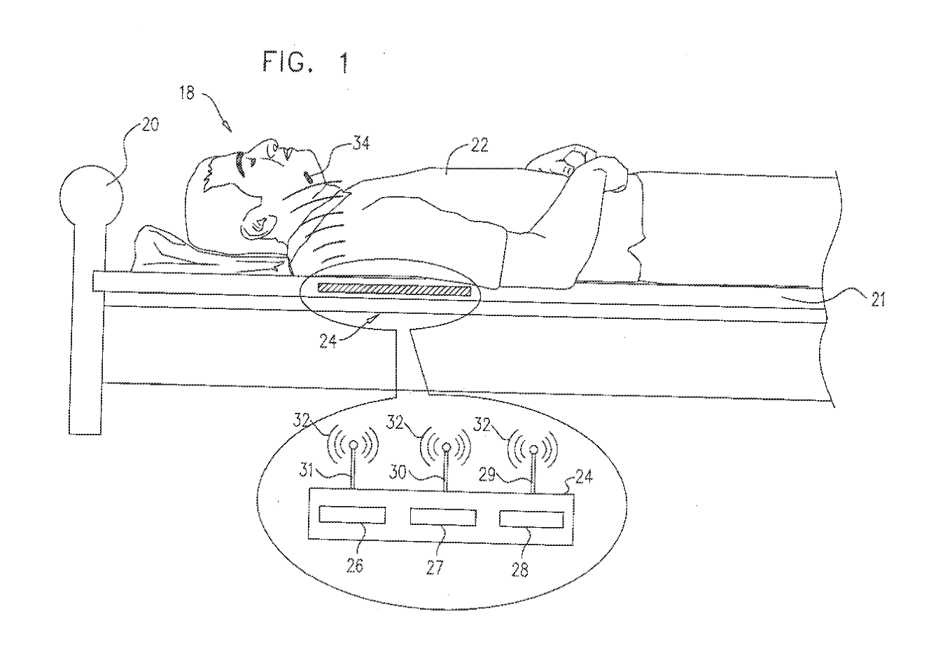

Reference is now made to FIG. 1, which is a schematic illustration of a bed 20 and a subject 22 lying supine on a mattress 21 of bed 20. Typically, the subject is sleeping. A system 18 is provided, which comprises an external device 24 and a sublingual implant 34, typically implanted at an anatomical site in the vicinity of sublingual muscles. Except for differences as noted, sublingual implant 34 is generally as described in U.S. patent application Ser. No. 12/946,246 to Gross, filed Nov. 15, 2010, which is incorporated herein by reference, in accordance with some applications of the present invention.

In further applications, system 18 comprises an external device 24 and a phrenic nerve implant 33 (not shown), typically implanted at an anatomical site in the vicinity of the phrenic nerve.

External device 24 comprises a control unit 26, a breathing sensor 28 and at least one power supply unit 27. External device 24, in accordance with some applications of the present invention, is placed in proximity to the subject, typically by being incorporated into subject's mattress 21. Alternatively, the external device can be placed anywhere near the subject, such that sublingual implant 34 receives signals from the external device. In some applications, external device 24 is placed in a chest band, in another article of clothing of the subject (e.g., a cap), or at another site near the subject. One or more antennas 29, 30, 31 of external device 24 are typically configured to send signals to the implant, as described hereinbelow. The configuration of antennas 29, 30, and 31 as shown in FIG. 1 are for illustrative purposes only. Typically, the antennas are not configured to be positioned in a straight line. In a further application, the antennas are not aligned in a same direction, e.g., the antennas are mutually perpendicular.

External device 24 is typically always powered on and, in some applications, does not have an on/off switch. In other applications, external device 24 is in standby mode, powering on to its active mode when the breathing of subject 22 is detected. In some applications, external device 24 is coupled to sensors, e.g., pressure sensors (not shown), to detect when subject 22 is lying down on mattress 21. The sensors are configured to communicate with external device 24, resulting in the powering on of external device 24 to its active mode in response to the lying down of subject 22.

In some applications, in which external device 24 is not continuously in powered on and active mode, e.g., when external device 24 is either completely powered off or in a stand-by mode, the device, when turned on, remains in the powered-on mode for a set duration, such as 5-120 minutes (e.g., 30 minutes). In other applications, the duration for which the external device remains powered-on can be configured to be a desired length of time.

In some applications, in which external device 24 is not continuously in powered on and active mode, e.g., when external device 24 is either completely powered-off or in a stand-by mode, the device powers on in response to the detection of a sleep-related breathing rate by breathing sensor 28. In further applications, external device 24 is configured to power-down in the morning, when breathing sensor 28 detects waking levels of breathing, or other indications of subject 22 being awake.

In some applications, breathing sensor 28 has a separate powered cycle than the other components of external device 24. For example, breathing sensor 28 may be powered separately from the other components in the external device.

Power supply unit 27 is configured to wirelessly charge sublingual implant 34, typically via induction, but other methods known in the art may also be used. Power supply unit 27 allows sublingual implant 34 to be constantly in a powered-on mode, typically without concern for draining batteries of the sublingual implant. In further applications, power supply unit 27 is configured to wirelessly charge phrenic nerve implant 33, allowing phrenic nerve implant 33 to be constantly in a powered-on mode, typically without concern for draining batteries of the implant.

When powered-on constantly in its active mode, or in other applications, when the external device is triggered by an event to power-on, external device 24 sends signals to sublingual implant 34 during or shortly after the event (for example, an abnormal breathing episode). In further applications, external device 24 sends signals to phrenic nerve implant 33 during or shortly after the event (for example, an abnormal breathing episode).

Typically one or more antennas 29, 30, 31 are configured to be in communication with power supply unit 27 to wirelessly power either sublingual implant 34 or phrenic nerve implant 33. In some applications, at least two antennas 29 and 31 are configured to send signals such that regardless of the position of subject 22, a sufficient amount of power will be typically received by sublingual implant 34. In other applications, at least two antennas 29 and 31 are configured to send signals such that regardless of the position of subject 22, a sufficient amount of power will be received by phrenic nerve implant 33. In some applications, the antennas are placed in a triangular formation. In some applications, the antennas are mutually-perpendicular antennas.

In some applications, antenna 30 is also configured to send power to sublingual implant 34 and/or phrenic nerve implant 33, either in lieu of, or in addition to, antennas 29 and 31.

Typically, the breathing sensor senses respiration, and control unit 26 sends signals 32 responsive thereto to sublingual implant 34, typically implanted in a muscle (e.g., the genioglossus muscle). In other applications, sublingual implant 34 is implanted on, at, or near a nerve that innervates a sublingual muscle of the subject. In some applications, sublingual implant 34 is implanted at or near the glossopharyngeal nerve. In further applications, sublingual implant 34 is implanted at or near the hypoglossal nerve. In some applications, a nerve cuff is used to stimulate a nerve in the vicinity of a sublingual muscle of the subject.

In some applications, external device 24 sends signals 32 to phrenic nerve implant 33 to stimulate the phrenic nerve or surrounding muscle for a pre-determined length of time, or in a particular pattern, or both, over the course of the sleep cycle of subject 22. Typically, phrenic nerve stimulator 33 is coupled directly to the phrenic nerve (not shown). In further applications, phrenic nerve implant 33 is implanted in a vein, and provides transvenous phrenic nerve stimulation.

External device 24 typically sends signals 32 to sublingual implant 34, which drive the sublingual implant to stimulate a sublingual muscle or nerve, as described hereinabove. The signals are typically sent to the implant at a particular temporal point in the subject's breathing pattern. In some applications, the temporal point is determined by breathing sensor 28. In some applications, the sublingual implant directly or indirectly stimulates a nerve that innervates a sublingual muscle, the stimulation being configured to cause the nerve, in response, to stimulate the sublingual muscle. In further applications, the signals are sent to the implant at a particular point in time, such that stimulation of the anatomical site during at least a portion of an expiration period identified by the external device is withheld, e.g., there is no stimulatory signal provided by external device 24 during expiration.

In further applications, external device 24 typically sends signals 32 to phrenic nerve implant 33, which drive the phrenic nerve implant to stimulate breathing muscles. The signals are typically sent to the implant during a particular, predesignated portion of the subject's breathing pattern. In some applications, the phases of the breathing pattern are determined in real-time by breathing sensor 28. In some applications, phrenic nerve implant 33 directly or indirectly stimulates a nerve that innervates a breathing muscle, the stimulation being configured to cause the nerve, in response, to stimulate the muscle.

The stimulation of muscle is typically in a plurality of successive periods of respiratory inspiration of subject 22, and, in some applications, even in the absence of a detection of apnea by external device 24.

For some applications, signals 32 are sent to the implant to cause the implant to stimulate the muscle and/or nerve at a non-inspiration point, or at multiple points during a respiratory cycle.

In some applications, external device 24 sends signals 32 to sublingual implant 34, or in some applications, to phrenic nerve implant 33, to stimulate the muscle and/or nerve for a pre-determined length of time, or in a particular pattern, or both, over the course of the sleep cycle of subject 22. In some applications, periods of no stimulation by sublingual implant 34, and in some applications, phrenic nerve implant 33, are provided, during the course of the sleep cycle, to prevent over-stimulating a muscle and/or nerve (e.g., the genioglossus muscle).

Typically, signals 32 are provided to sublingual implant 34, and in some applications, phrenic nerve implant 33, during at least 30% or at least 50% of the respiratory periods over the course of a ten-minute period, even in the absence of a detection of apnea by external device 24.

Typically, external device 24 is typically configured to only provide signals 32 to the sublingual implant 34, or in some applications, phrenic nerve implant 33, to stimulate a muscle and/or nerve when the subject 22 is sleeping and in the vicinity of external device 24 (e.g., in mattress 21 of bed 20, or in another item near bed 20).

In some applications, breathing sensor 28 is coupled to mattress 21. In further applications, breathing sensor 28 is housed within mattress 21. Breathing sensor 28 typically comprises a piezoelectric crystal, a strain gauge a pressure sensor, an accelerometer, an optical sensor, an audio sensor, a video imager, and/or a cardiac sensor, or combinations thereof, to detect, for example, breathing-related motions, snoring, electrical activity, and/or other indications of the subject's breathing. The one or plurality of sensors provide information regarding, but not limited to, the subject's airway pressure, snoring sounds and motions, mechanical motion associated with respiration, and/or electrical activity associated with respiration. In addition, other sensors known in the art (e.g., a blood oxygen saturation sensor) may assess the operation of external device 24 and implant 34, and support closed-loop control of external device 24 and implant 34, and/or implant 33.

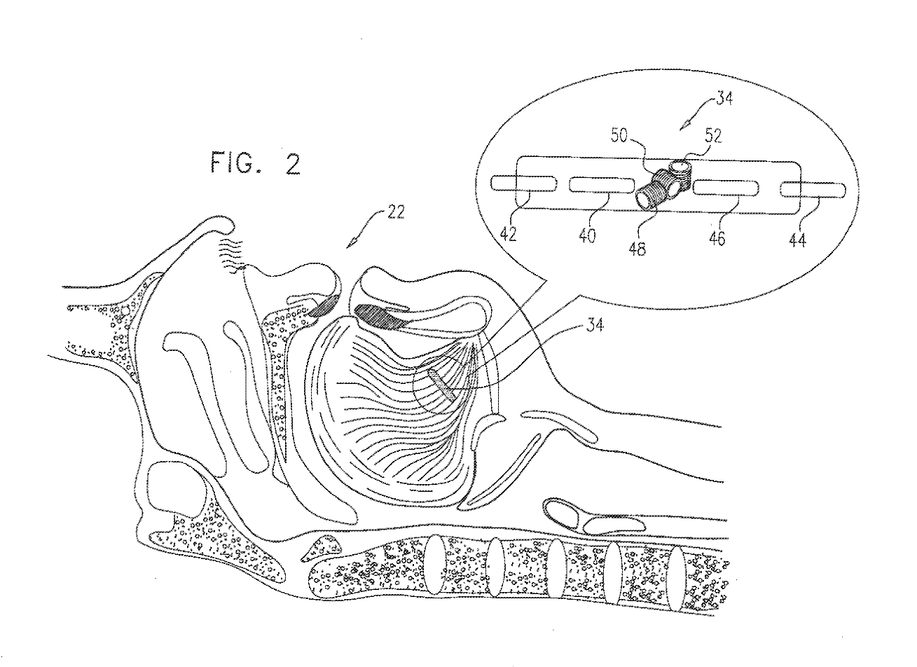

Reference is now made to FIG. 2, which is a schematic illustration of sublingual implant 34 in subject 22, in accordance with some applications of the present invention. Sublingual implant 34 shown in FIG. 2 is typically the sublingual implant described hereinabove with reference to FIG. 1. Sublingual implant 34, shown enlarged, contains a wireless receiver 40, two or more electrodes 42 and 44, and circuitry 46, coupled to the electrodes. In some applications, sublingual implant 34 has one or more power receiving antennas 48, 50 and 52. In further applications, sublingual implant 34 has one or more power receiving antennas oriented in x, y and z directions, configured such that regardless of the position of subject 22, a sufficient amount of power will be received by the antenna. In some applications, the antennas are mutually-perpendicular antennas. In some applications the mutually-perpendicular antennas comprise three coils, each oriented in one of the x, y and z directions.

The implant and its components are encapsulated using techniques known in the art. Material for the electrodes may be chosen based on numerous criteria, as are known in the art, including tissue response, allergic response, electrode-tissue impedance, and radiographic visibility.

Circuitry 46 is typically configured to drive electrodes 42 and 44 to apply current, typically as a train of pulses, to a sublingual site of subject 22 (as described hereinabove), in response to signals 32 from external device 24, in a plurality of successive periods of inspiration of the subject. Typically, signals 32 are generated substantially for the duration of any given sleep cycle, even in the absence of a detection of apnea by the external device. Alternatively, signals 32 are generated for only a portion of the sleep cycle, e.g., for at least 30% or at least 50% of the cycle.

It is to be noted that the sublingual implant's particular location in the muscle is shown by way of illustration and not limitation.

In some applications, sublingual implant 34 includes an implant sensor (not shown), such as a vibration sensor sensitive to snoring vibrations, or a motion sensor. The implant sensor allows the implant to operate independently of, or in conjunction with, external device 24, optionally providing feedback to the external device. In other applications, one or more other implant sensors are included in the implant to be used independently by the implant, or in conjunction with external device 24.

In some applications, the operation parameters of sublingual implant 34—e.g., duty cycle, frequency, pulse duration or amplitude, of the sublingual implant's current, can be altered in response to how effective or painless the current generated by the implant is, and/or whether the implant disrupts the subject's sleep. This may be done with a wand (not shown), or with an input unit such as a keypad (not shown) on external device 24.

Feedback parameters for determining the efficacy and/or efficiency of the implant are obtained via receiving information from subject 22 himself, by way of sensors in external device 24, or in some applications, by feedback sensors (such as vibration or acceleration sensors, not shown) in sublingual implant 34.

It is noted that the number of sublingual implants shown in the figures is by way of illustration and not limitation. One or more of the implants are typically injected into the subject in a minimally-invasive manner, in an outpatient procedure. Typically, this is done through the lumen of a needle (not shown) using aseptic technique and with local anesthesia, as is known in the art. Other surgical procedures known in the art may also be used to place the implant in a muscle.

For some applications, one or more implants 34 are configured to work in conjunction with other implants or independent of each other and/or external device 24.

Typically, sublingual implant 34 is also configured to wirelessly receive power from external device 24, e.g., via a coil, e.g., coil 48, coupled to circuitry 46. Other applications may use any one of, or a combination of, forms of wireless energy transfer, such as inductive coupling, RF, ultrasound, or other forms of wireless energy transfer.

In some applications, sublingual implant 34 is continuously provided with the necessary power to stimulate the muscle or nerve via power supply unit 27, external device 24, or some other external device (not shown), in close enough proximity to wirelessly transfer power to the implant, as described hereinabove with reference to FIG. 1.

In some applications, sublingual implant 34 can temporarily store electrical power, e.g., by use of a battery or high-capacity capacitor (not shown), coupled by a wire (not shown) to the implant, or disposed within the implant.

In some applications, sublingual implant 34 has an internal battery that is periodically charged by power supply unit 27 or some other external device (not shown), in close enough proximity to wirelessly transfer power to the implant from power supply unit 27, or a different power source (not shown).

For some applications, external device 24 operates substantially throughout the night, e.g., causing sublingual implant 34 to drive current into the sublingual muscle and/or sublingual nerve during most respiration cycles. Alternatively, external device 24 determines if the subject is sleeping, and only drives implant 34 during these periods (e.g., only during inspiration during sleep). Further alternatively, external device 24 determines if the subject is snoring, or exhibiting another respiration problem, and only drives the implant during such periods. Other operating parameters have been described hereinabove with reference to FIG. 1. These parameters are described by way of example and illustration but not limitation; other parameters for powering and monitoring sublingual implant 34 and external device 24 are also included within the scope of the present invention.

The aforementioned description of FIG. 2, described hereinabove with relation to sublingual implant 34 is also applicable to phrenic nerve implant 33 (not shown) with the difference described below: phrenic nerve implant is typically implanted in the vicinity of the phrenic nerve (e.g., near the phrenic nerve or surrounding or otherwise in contact with the phrenic nerve).

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for treating obstructive sleep apnea, comprising:
an external device, comprising:
a breathing sensor configured for placement outside of a body of a subject, and to sense activity associated with breathing of the subject; and
a control unit, coupled to the breathing sensor, configured to generate a signal in response to the sensed activity; and
an implant, comprising:
a wireless receiver for receiving the signal;
two or more electrodes; and
circuitry coupled to the electrodes, the circuitry configured to drive the electrodes to apply current to an anatomical site of the subject in response to the signal, in a plurality of successive periods of inspiration of the subject, even in the absence of a detection of apnea by the control unit.

2. The apparatus according to claim 1, wherein the implant is configured to be implanted sublingually.

3. The apparatus according to claim 1, wherein the implant is configured to stimulate a phrenic nerve of the subject.

4. The apparatus according to claim 1, wherein the implant is configured to stimulate breathing muscles associated with a phrenic nerve of the subject.

5. The apparatus according to claim 1, wherein the external device is configured to be coupled to a mattress.

6. The apparatus according to claim 1, wherein the external device is configured to be coupled to an article of clothing.

7. The apparatus according to claim 1, wherein the two or more electrodes are configured to be coupled to a sublingual muscle of the subject.

8. The apparatus according to claim 1, wherein the implant comprises at least two antennas that are not aligned in a same direction, and wherein the implant is configured to receive the signal via the at least two antennas.

9. The apparatus according to claim 1, wherein the breathing sensor is configured to sense a parameter selected from the group consisting of: airway pressure, snoring sounds, snoring motions, mechanical motion, and electrical activity associated with respiration.

10. The apparatus according to claim 1, wherein the implant is operative to vary at least one parameter of the applied current, the parameter selected from the group consisting of: a frequency of pulses of the current, an amplitude of pulses of the current, and duration of pulses of the current.

11. The apparatus according to claim 1, wherein the implant comprises an implant sensor configured to provide feedback to the external device.

12. The apparatus according to claim 1, wherein the control unit is configured to generate the signal during respective inspiratory phases of at least 30% of respiratory periods of the subject in a ten-minute period.

13. The apparatus according to claim 1, wherein the external device comprises a power supply unit, which is configured to supply power wirelessly to the implant.

14. The apparatus according to claim 1, wherein the implant comprises at least two antennas that are not aligned in a same direction, and wherein the implant is configured to receive the power via the at least two antennas.

15. The apparatus according to claim 1, wherein the two or more electrodes are configured to be coupled to a sublingual nerve of the subject.

16. The apparatus according to claim 15, wherein the implant comprises a nerve cuff to which the electrodes are coupled.

17-42. (canceled)
